(19) United States
(12) Patent Application Publication
(10) Pub. No.: US 2024/0216099 A1
(43) Pub. Date: Jul. 4, 2024

(54) COMPOSITE FLEXIBLE MATERIALS FOR PORTABLE SURGICAL SYSTEMS

(71) Applicant: SURGIBOX INC., Cambridge, MA (US)

(72) Inventors: Debbie Lin Teodorescu, Cambridge, MA (US); Stephen Michael Okajima, Boston, MA (US); Gelu Comanescu, Washington, DC (US)

(73) Assignee: SURGIBOX INC., Cambridge, MA (US)

(21) Appl. No.: 18/402,613

(22) Filed: Jan. 2, 2024

Related U.S. Application Data
(63) Continuation-in-part of application No. 17/055,180, filed on Nov. 13, 2020, now abandoned.
(60) Provisional application No. 62/672,070, filed on May 16, 2018.

(51) Int. Cl. A61B 46/00; A61F 13/36

(52) U.S. Cl. CPC A61B 46/40; A61F 13/36

(57) ABSTRACT

A composite-material for use in portable surgical systems is disclosed. The composite material may comprise two layers of a first material and one layer of a second material. The layer of the second material may comprise a plurality of holes and may be disposed between the layers of the first material. The composite-material may further include a series of bonding regions extending, from one layer of the first material to the other layer of the first material, through the plurality of holes of the layer of the second material. The bonding regions may seal the holes of the layer of the second material such as to form an impenetrable composite-material layer even when the layers of the first material are not impermeable. The bonding regions may cause the layers to attach to each other. The composite-material may be used to make a sterile sleeve of the portable surgical system.

### CROSS REFERENCE TO RELATED APPLICATION

This application is a continuation in part of and claims priority from U.S. Nonprovisional patent application Ser. No. 17/055,180 filed on Nov. 13, 2020 and titled “Sterile Sleeves for Portable Surgical Systems” (a national stage application of International Patent Application No. PCT/US19/32148) which are hereby incorporated by reference for all purposes as if fully set forth herein. This application claims priority from and the benefit of U.S. Provisional Patent Application No. 62/672,070 filed on May 16, 2018 and titled “Method of joining two dissimilar materials to retain an impermeable barrier”, and U.S. Provisional Patent Application No. 62/670,891 filed on May 14, 2018 and titled “Sterile sleeve for sterile procedures and re-entry” which are hereby incorporated by reference for all purposes as if fully set forth herein.

### BACKGROUND OF INVENTION

### I. Field of the Invention

Exemplary embodiments of the present invention relate to a portable surgical system for regulating intra-operative environments over surgical sites, to a sterile sleeve for entry in a sterile environment, and to methods for implementing and using the same.

### II. Discussion of the Background

Over 25% of the global disease burden requires surgical therapy, which could prevent over 18 million deaths per year. These range from obstetric complications to traumas to infections to cancer and beyond. Yet 2 billion people have no meaningful access to safe surgical care, and 2-3 billion more have access only to unsterile surgeries in contaminated environments, leading to disproportionate rates of surgical infections. Innovations in this field typically focus upon making operating rooms and operating room ventilation systems more mobile, such as in tent format. However, such systems remain costly to purchase and to maintain. Moreover, such systems are difficult to transport rapidly to remote areas. At the same time, over 85,000 medical providers are infected by patient bodily fluids annually, with 90% of infected providers worldwide having been exposed while working in low-resource settings. While personal protective equipment mitigates these risks to some extent, there is a definite trade-off between the level of protection and both the cost as well as the user comfort, which is well-documented to correspond to user compliance.

As is commonly seen in the medical and bio-medical fields, most interactions with sterile environments are performed through Personal Protective Equipment such as surgical gowns, gloves, drapes, and other barriers. These barriers are not only necessary for maintaining a sterile environment for the operator to work within, but also are crucial for maintaining the safety of the operator from potentially harmful exposure to hazardous substances.

Surgical gowns remain a staple for surgeons to maintain both sterility and safety when performing a procedure. While these gowns are effective, with varying levels of protection, they are designed with a focus on isolating the operator from the sterile field to ensure that no contaminants from the operator enter the field. This is only sufficient from a protective standpoint, however, as other means are necessary to maintain sterile the field of interest. Other devices such as glove boxes require large amount of resources, require complicated setups, or they provide an inflexible point of entry that can hinder certain operations. The current options for maintaining a safe/sterile environment often require a substantial amount of resources or an established infrastructure in order to be used effectively.

International PCT application number PCT/US2017/042266 (publication number WO/2018/014003) filed on Jul. 14, 2017 and titled “Ultraportable System For Intraoperative Isolation and Regulation of Surgical Site Environment”, which is hereby incorporated by reference for all purposes as if fully set forth herein, addresses some of the challenges of patient and provider intraoperative exposure to infectious risks by implementing an ultraportable, self-contained, passive and active, bilateral barrier against exchange of contaminants between incisions and the greater surgical area.

The application herein addresses the need to have a comfortable, user friendly, inexpensive, and effective method for creating and safely interacting with a sterile environment.

The above information disclosed in this Background section is only for enhancement of understanding of the background of the invention and therefore it may contain information that does not form any part of the prior art.

### SUMMARY OF THE INVENTION

Exemplary embodiments of the present invention provide a portable surgical system provided with one or more sleeves for regulating intra-operative environments over surgical sites.

In an exemplary embodiment, it is disclosed a sterile sleeve for use with the enclosure of a portable surgical system so as to provide a resource efficient, comfortable, and easy to use point of entry. The sleeve may be made of a composite material including at least one plastic. The composite material of the sterile sleeve may be impermeable. The sleeve may adopt a combination of effective qualities from the combined materials that will allow the invention to achieve the desired level of protection (e.g. an AAMI level 4 protection from testing, ATSM F 1671:2003).

In an exemplary embodiment, the portable surgical system comprises a sterile enclosure and one or more sterile sleeves made of a composite-material.

The composite-material may comprise one or more layers of a first material; one or more layers of a second material; and a series of intermixed regions between the layer of the first material and the layers of the second material. The intermixed regions acts as a bond between the layers. The intermixed regions may be formed by applying heat and/or pressure to the layers such as to maintain the continuity and impermeability of the composite layer. At least one of the first and the second materials may be a thermoplastic. The intermixed regions may be the result of the penetration of heated material of one layer into the pores or cavities of another layer. The composite-material may be impermeable to a set of fluids, gasses, and substances comprising one or more of the following: blood, plasma, bacteria, viruses, dust particles, water, human and animal cells, and organic matter. In an exemplary embodiment, the composite-material above may include two layers of the first material and one layer of the second material. In another exemplary embodiment, the composite-material above may include one layer of the first material and one layer of the second material.

The composite-material may include two layers of a first material and one layer of a second material. The layer of the second material may include a plurality of holes. The layer of the second material may be disposed between the layers of the first material. The composite-material may include a series of bonding regions extending from one layer of the first material to the other layer of the first material through the plurality of holes of the layer of the second material. The bonding regions may substantially seal the holes of the layer of the second material such as to form a substantially impenetrable composite-material layer even when the layers of the first material are not impermeable. The bonding regions are causing the layers to attach to each other.

The sterile sleeves may comprise a substantially conically shaped layer made of the composite-material and configured to cover the length of an user's forelimb. The conically shaped layer may include a first opening and a second opening. The first opening may be configured to enable an user to insert a hand in the sleeve. The second opening may be attached to a port of the enclosure such as to enable the hand of the user to access the inner of the enclosure via the port. The sleeve may further include a mechanism configured to secure the user's wrist in place with respect to the sleeve such that a good seal and a personalized fit can be established. The sleeve may further include a sterile cover configured to cover the first opening such as to maintain the sterility inside the enclosure and configured to be removed by the user prior to accessing the port.

The sleeve may comprise two dissimilar materials, one of which may be a thermoplastic. The first layer may be chosen for its comfort and ease of use, and the second layer may be chosen for its impermeability.

It is to be understood that both the foregoing general description and the following detailed description are exemplary and explanatory and are intended to provide further explanations of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention, and together with the description serve to explain the principles of the invention.

FIG. 1 is a side view of an inflated portable surgical enclosure adhered to the patient's torso surgical site via incise drape, with air inflow from air supply in the enclosure side closest to patient feet, directed in cranial longitudinal direction over the patient's surgical site.

FIG. 2 is a top view of the inflated portable surgical enclosure from FIG. 1 with two users working via arm ports in the operating-section on the torso surgical site, and two users working via arm ports in instrument-section.

FIG. 3A is a side view of an alternate embodiment of the surgical enclosure and frame, in which the rigid frame fully supports the enclosure with frame attachment to each of the sides defining the top of the enclosure. The enclosure extends circumferentially around the patient torso.

FIG. 3B shows an oblique perspective view of the frame and plastic enclosure shown in FIG. 3(a).

FIG. 4 shows a schematic view of a sterile sleeve included in a portable surgical system and configured for the sterile access of the interior of the enclosure.

FIG. 5A shows a schematic view of a sleeve material including two layers.

FIG. 5B shows a schematic view of a sleeve material including three layers.

FIG. 6A shows a schematic view of a first stage for a method for making a sleeve layer out of three individual layers.

FIG. 6B shows a schematic view of a second stage for a method for making a sleeve layer out of three individual layers.

FIG. 7A shows a cross section of three layers prior to fusion.

FIG. 7B shows a schematic view of cross sections of three sleeve layers attached to each other by bonding structures.

FIG. 7C shows a schematic view of cross sections of three sleeve layers attached to each other by bonding structures.

FIG. 7D shows a schematic view of cross sections of three sleeve layers attached to each other by bonding structures.

FIG. 7E shows a schematic view of cross sections of three sleeve layers attached to each other by bonding structures.

FIG. 8 shows an exemplary embodiment for a method for making a sleeve layer out of three individual layers.

FIG. 9A shows a cross section of the sleeve layers in FIG. 8 before the individual layers are fused to each other.

FIG. 9B shows a cross section of the sleeve layers in FIG. 8 after the individual layers are fused to each other.

FIG. 10 shows an exemplary embodiment in which the outer layers are fused together around the inner layer and along an edge of the sleeve material.

FIG. 11A shows schematically a view of a method of attaching two sleeve layers to each other along their edges.

FIG. 11B shows schematically another view of a method of attaching two sleeve layers to each other along their edges.

FIG. 12A shows schematically a view of a second method of attaching two sleeve layers to each other along their edges.

FIG. 12B shows schematically another view of a second method of attaching two sleeve layers to each other along their edges.

FIG. 13A shows a schematic view for a first stage of a method for making a sleeve layer out of two individual layers.

FIG. 13B shows a schematic view for a second stage of a method for making a sleeve layer out of two individual layers.

FIG. 14A shows a cross section of two layers prior to fusion.

FIG. 14B shows a schematic view of cross sections of two sleeve layers attached to each other by bonding structures.

FIG. 14C shows a schematic view of cross sections of two sleeve layers attached to each other by bonding structures.

FIG. 14D shows a schematic view of cross sections of two sleeve layers attached to each other by bonding structures.

FIG. 14E shows a schematic view of cross sections of two sleeve layers attached to each other by bonding structures.

FIG. 15A shows an exemplary embodiment of a sleeve.

FIG. 15B shows a cross section of an exemplary embodiment of a sleeve.

FIG. 16A shows the first stage of two stages of a method of accessing the enclosure via a sleeve.

FIG. 16B shows the second stage of two stages of a method of accessing the enclosure via a sleeve.

### DETAILED DESCRIPTION OF THE ILLUSTRATED EMBODIMENTS

The invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure is thorough, and will fully convey the scope of the invention to those skilled in the art. In the drawings, the size and relative sizes of layers and regions may be exaggerated for clarity. Like reference numerals in the drawings denote like elements.

It will be understood that when an element or layer is referred to as being “on” or “connected to” another element or layer, it can be directly on or directly connected to the other element or layer, or intervening elements or layers may be present. In contrast, when an element or layer is referred to as being “directly on” or “directly connected to” another element or layer, there are no intervening elements or layers present. It will be understood that for the purposes of this disclosure, “at least one of X, Y, and Z” can be construed as X only, Y only, Z only, or any combination of two or more items X, Y, and Z (e.g., XYZ, XY, YY, YZ, ZZ).

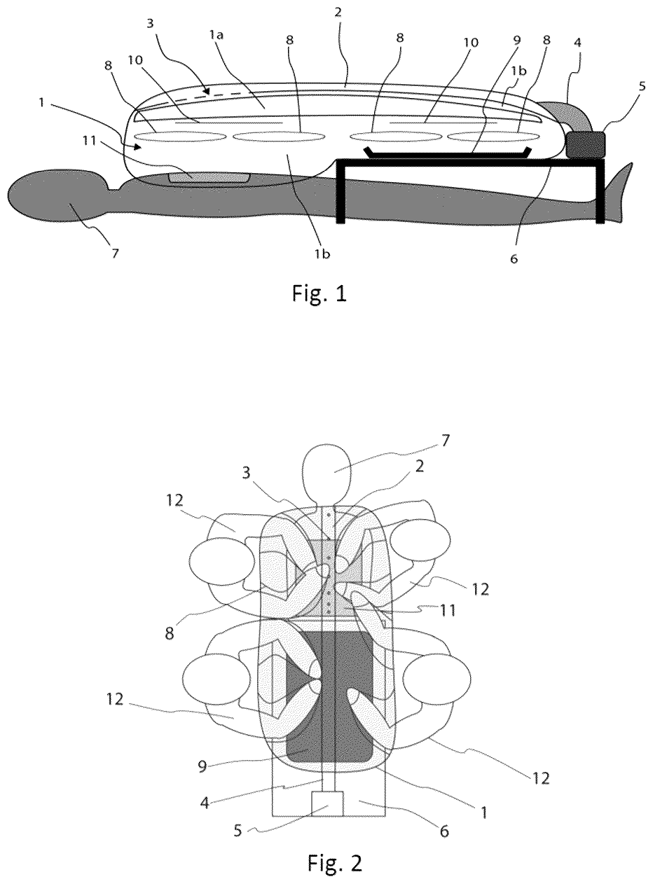

FIG. 1 illustrates an embodiment of a portable surgical system as the one presented in international patent application PCT/US2017/042266 (pub. no. WO/2018/014003 titled “Ultraportable System for Intra-Operative Isolation and Regulation of Surgical Site Environments”, filed on Jul. 14, 2017) which is incorporated hereinafter in its entirety as if fully set forth herein.

The portable surgical system includes a flexible plastic enclosure 1 configured to be supplied with air via an environmental control system 5. The enclosure 1 may be adhered to a surgical site of a patient 7 via an incise drape 11 as shown in FIG. 1. The portable surgical system may include a plurality of ports, such as arm ports 8 and material ports 10 shown in FIGS. 1 and 2. The portable surgical system may include a plurality of integrated, cuffed sleeves in the arm ports 8. The ports 8 provide users 12 with access to the inside of the enclosure, as shown in FIG. 2. The material ports 10 may be used to move a surgical tray 9 to the inside of the enclosure 1 prior to the surgical procedure. The portable surgical system may further include an instrument tray holder 6 which may be placed around the legs of the patient 7.

When set up, the surgical enclosure may comprise one or more top view panels of optically-clear plastic 1a, such as polyvinyl chloride. The remainder of the surgical enclosure sides may comprise a flexible, impermeable plastic, such as low-density polyethylene 1b.

The surgical enclosure 1 may include incise drapes 11 of different shapes and sizes and may be disposed at different positions on the surgical enclosure such as to fit the needs of different types of medical procedures. The bottom corners of the surgical enclosure may include straps for securing the enclosure to the patient or to the operating table for additional stability.

The environmental control system 5 may include an air supply system for supplying air to the enclosure via an inlet, such as a flexible tubing 4 and/or a valve. The air supply system may include a HEPA filter, a fan, a manual pump, a battery, a control section, and a sterile flexible tubing. The HEPA filter may be changeable and customizable such that it provides one or more other controls based on procedural need, such as humidity modulator filter, gas content with supply of medical gases, or temperature modulator with heat/cold sinks. The environmental control system is capable of enacting such pre-selected controls required for a given procedure, such as: HEPA filtration, pressure control (e.g. positive pressure inside the enclosure), humidity modulation, heating or cooling, or change of gas composition. The portable surgical system may be configured such that filtered air is blown or passed through a longitudinal tubular valve with walls of flexible, collapsible plastic such as polyethylene 2 and through a manifold with perforations 3. The filtered air may be blown such as to cause an essentially uniform laminar air flow onto the surgical site and through the enclosure.

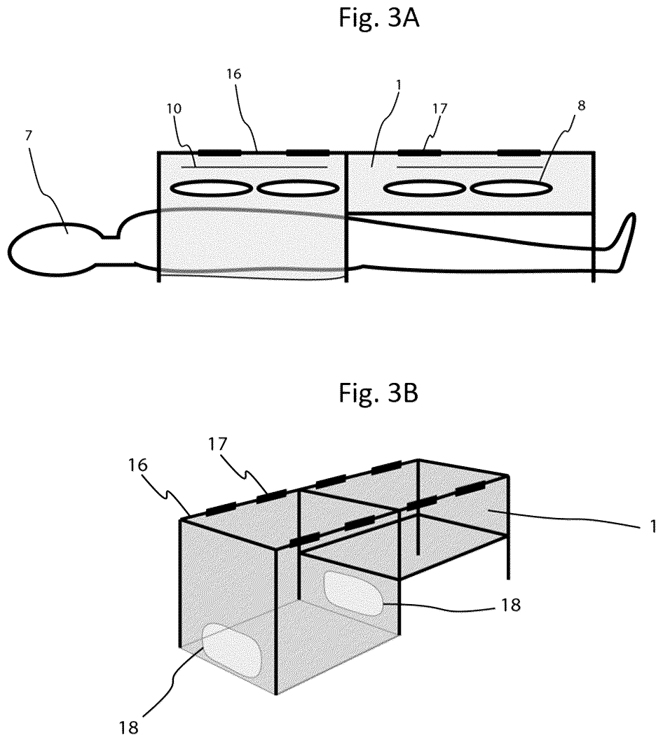

The surgical enclosure 1 may be supported by a system of frames disposed inside or outside the enclosure 1, such as the frames described with reference to FIGS. 3-8 in international PCT patent application publication no. WO/2018/014003. The system of frames is configured to provide support to the enclosure 1 in the event of a sudden pressure loss.

In an exemplary embodiment the surgical enclosure may be disposable, such as the enclosure 1 shown in FIG. 1. In an exemplary embodiment the surgical enclosure may be supplied folded like a surgical gown.

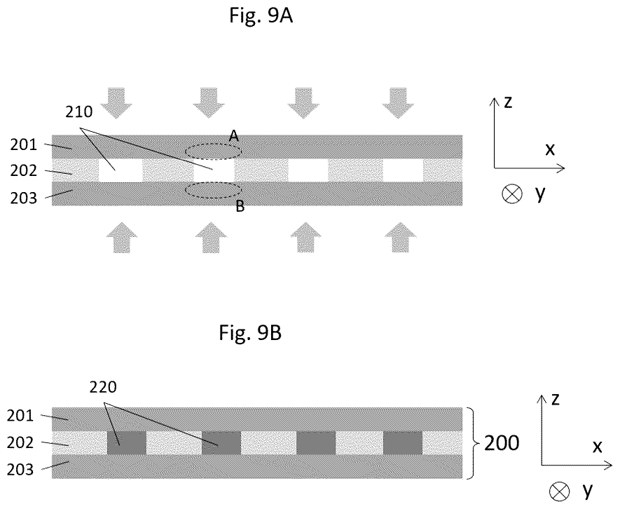

FIG. 3A and FIG. 3B illustrate a side view and a perspective view, respectively, of a second preferred embodiment of the portable surgical system. In the second preferred embodiment the portable surgical system includes an incise drape-less surgical enclosure 1 wherein the operating-section of the patient is placed inside the enclosure and wherein the bottom of the enclosure remains continuous with the sides at the level of the instrument-section. In the exemplary embodiment shown by FIG. 3A and FIG. 3B the enclosure 1 encloses patient's torso and the operating-section, thereby isolating the operating-section from the outer environment. The enclosure 1 may be supported via a system of frames, such as the rigid frame 16, illustrated in FIGS. 3A and 3B (and described in more detail with reference to FIG. 9 and in international patent application WO/2018/014003). The cranial end of the operating-section 18 as well as the interface with the instrument-section 18 may be secured against the patient via integrated straps.

The various embodiments of the portable surgical system may include a surgical enclosures, wherein the enclosures may further include a plurality of ports. The enclosure may include materials ports, instrument ports, and arm ports.

The materials port, such as 10 shown in FIGS. 1, 3A and 3B, allow the instrument tray 9 and instruments to be moved into the enclosure 1 prior to the procedure. Additionally, the materials port allows materials to be moved in and out of the enclosure throughout the surgical procedure. The enclosure may include large ports, small ports, and ports of adjustable size. Large ports permit the moving of large items like the instrument tray, neonates, et cetera in and out of the enclosure. The ports may be opened and closed by various means, such as zippers, magnetic strips, hook-and-loop fasteners, flexible inflatable tubes compressed against one another, or other methods. The size of the ports may be adjusted by various means, such as a zipper slider that slides over the zipper teeth rows thereby adjusting the size of the port. In addition to episodic access for large items, the ports can also provide ongoing access for lines, tubes, wires, and drains requiring access to external resources.

Ports may be configured such that items may be passed in or out of the enclosure without significant relative loss of enclosure volume or pressure, regardless of frame availability, because the Environmental Control System (e.g. a fan) can increase the gas inflow to match the outflow.

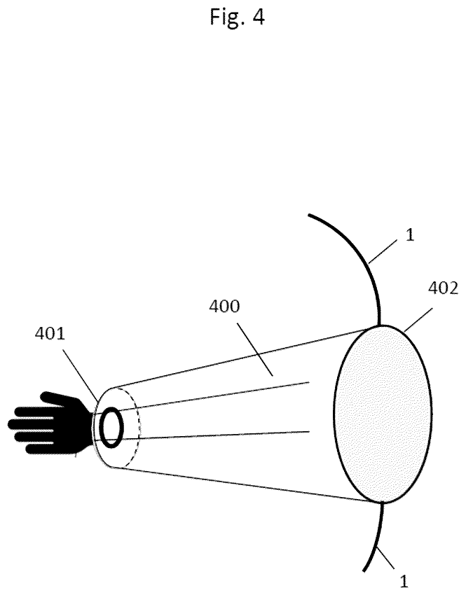

The arm ports 8, as shown in FIGS. 1, 2, and 3A and 3B allow access to the inside of the enclosure by either provider arms or augmenting instrumentation taking the place of arms such as laparoscopes or robots. The number of arm ports is dependent on procedural need. The preferred embodiments illustrated in FIGS. 1, 2, and 3A and 3B include four pairs of arm ports 8, two on each side of the enclosure 1. Depending on use scenario, the arm ports may take two major forms. The first form for the arm port is a simple opening in the side of the enclosure which seals reversibly against user arms. The second form for the arm port is a sleeve, as shown by 400 in FIG. 4. The sleeve may include a hollow cylinder or frustrated cone of material (e.g. fabric; rubber; plastic; or a composite/combination that may include fabric, plastic, surgical glove material, latex and other materials) that may taper toward the inside of the enclosure away from the wall. The sleeve end may be free or may incorporate a cuff of elastic material 401 to fit against the user wrist. In another exemplary embodiment the sleeve may end in a glove. The length of the sleeve is such as to permit ergonomic handoff of instruments among ports at contralateral ends of the system. At the other end, the sleeve is attached to the enclosure 1 at 402 (e.g. by sewing or heat).

The sleeve material may include one or more layers of materials, such as, but not limited to: fabric, rubber, thermoplastics or a combination that may include fabric, plastic, surgical glove material, latex, polyurethane, polycarbonate, acetal copolymer polyoxymethlene, acetal homopolymer polyoxymethylene, acrylic, nylon, polypropylene, polystyrene, or thermoplastics that are sufficiently non-brittle to act as a cloth-like material. In the following we describe (a) exemplary embodiments for which the sleeve material layer includes essentially three distinct layers, and (b) exemplary embodiments for which the sleeve material layer includes essentially two distinct layers.

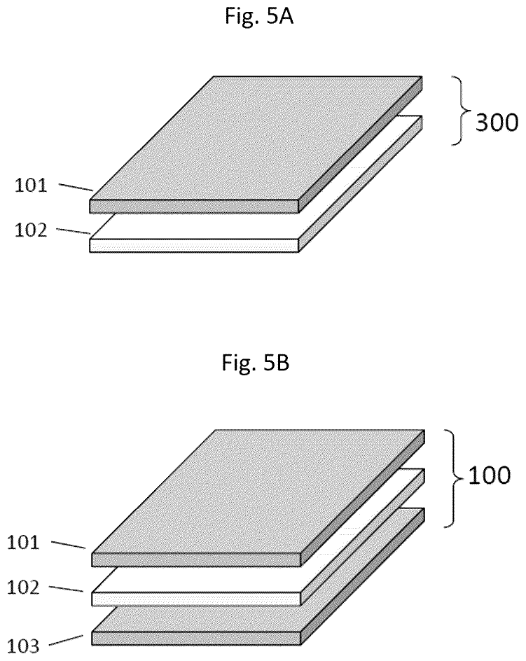

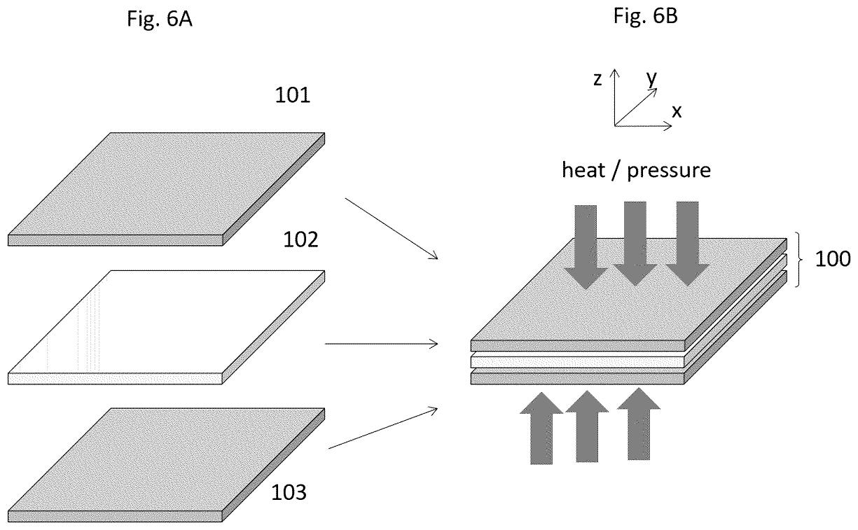

In an exemplary embodiment, the sleeve material layer 100 may include three distinct layers disposed over each other: layer 101, layer 102, and layer 103 (as shown in FIG. 5B). Layer 101 may be disposed to face the arms of the user, thereby touching the arms of the user during operation. Layer 103 may be disposed to face the interior of the flexible plastic enclosure, thereby being exposed to the environment inside the enclosure during operation. Layer 102 is disposed in between layers 101 and 103. Layers 101, 102, and 103 may be attached to each other as discussed hereinafter with reference to FIGS. 6 to 10.

Layers 101 and 103 may be configured to serve as external layers with focus on ergonomics, case of use, or functional properties. These layer's functional properties could include: material's ability to reduce heat retention, increase heat retention, wick up moisture, decrease friction, increase friction, or other properties that would benefit the comfort or functionality of the sleeve.

Layer 101 may be made of a porous material and/or may be configured such as to feel comfortable when touching operator's hand and such as to absorb/wick up moisture.

Layer 102 may be configured to maintain an impermeable barrier from the internal sterile environment and the external environment. Layer 102 may include on or more of the following materials: impermeable thermoplastics such as thermoplastic polyurethane, polycarbonate, acetal copolymer polyoxymethlene, acetal homopolymer polyoxymethylene, acrylic, nylon, polypropylene, polystyrene, or other thermoplastics that are sufficiently non-brittle to act as a cloth-like material.

Layer 103 may be made of a porous material and/or may be configured such as to create a comfortable feel when touching a patient and such as to absorb/wick up moisture from inside the enclosure.

Layers 101 and 103 may be made of the same materials, may have the same properties and composition, or may be made from the same type of material layer.

In another exemplary embodiment layers 101 and 103 may be made to maintain an impermeable barrier whereas layer 102 may be porous. Layers 101 and 103 may include on or more of the following materials: impermeable thermoplastics such as thermoplastic polyurethane, polycarbonate, acetal copolymer polyoxymethlene, acetal homopolymer polyoxymethylene, acrylic, nylon, polypropylene, polystyrene, or other thermoplastics that are sufficiently non-brittle to act as a cloth-like material. Layer 102 may be made of a material including pores. Layers 101, 102 and 103 may be attached or adhered to each other to form the sleeve layer material 100 by using various processes.

In an exemplary embodiment, a method of making a sleeve layer is described with reference to FIGS. 6A and 6B. In one embodiment, layer 102 may be an impervious thermoplastic whereas layers 101 and 103 may be made of a material including pores (which may not be impervious). Layers 101, 102, and 103 may be adhered together by applying heat, specific to the melting temperature of the inner thermoplastic layer 102, and sufficient pressure, such as to allow for the thermoplastic to infuse (e.g. via melting) within the pores of one or both outer materials 101 and 103. In this way, the inner impervious thermoplastic will remain continuous with itself while also becoming mechanically adhered within the pores of the outer materials 101 and 103, effectively creating a fitted bond. The layers 101, 102 and 103 may be adhered/bonded together by various means known by the skilled artisans, such as: applying heat, applying pressure, ultrasonic welding, laser welding, photochemical reactions (light induced), thermally induced reactions or any combination of them.

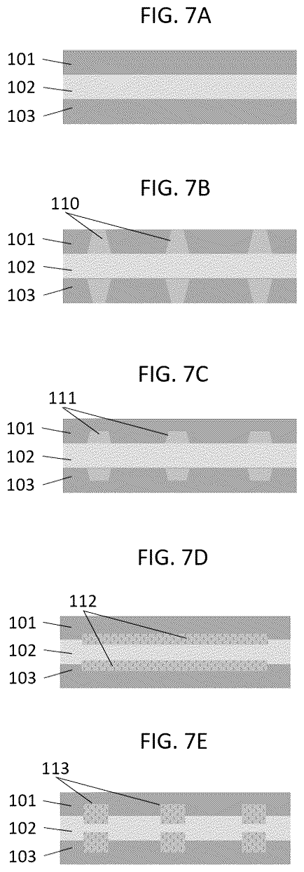

FIG. 7A shows a cross section of the layers prior to fusion. In an exemplary embodiment, the resulting fusioned layers may have a structure as qualitatively shown in FIG. 7B wherein the layer 102 has formed fusion protrusions 110 inside the porous layers 101 and 103, thereby creating a bond between layers 101, 102 and 103. The protrusions 110 completely penetrate through the layers 101 and 103. The fusion protrusions may act as separated bonding-regions between the layers. The fusion protrusions 110 may be arranged in two 2D arrays parallel with the layers. The first 2D array will be disposed essentially in the plane of layer 101 whereas the second 2D array will be disposed essentially in the plane of layer 103.

In an exemplary embodiment, the resulting fusioned layers may have a structure as qualitatively shown in FIG. 7C wherein the layer 102 has formed fusion protrusions 111 inside both layer 103 and 101, thereby creating a bond between layers 101, 102 and 103. The protrusions 111 reach inside layers 101 and 103 without completely penetrating them. The fusion protrusions may act as separated bonding-regions between the layers. The fusion protrusions 111 may be arranged in two 2D arrays parallel with the layers. The first 2D array will be disposed 350 essentially in the plane of layer 101 whereas the second 2D array will be disposed essentially in the plane of layer 103.

In an exemplary embodiment, the resulting fusioned layers may have a structure as qualitatively shown in FIG. 7D wherein the layers 101 and 102 intermix between them at the interface 101-102 over substantially the entire area of the layers, thereby forming an intermixed region 112 and creating a bond between layers 102 and 101. Similarly, the layers 103 and 102 intermix between them at the interface 103-102 over substantially the entire area of the layers, thereby forming an intermixed region 112 and creating a bond between layers 102 and 103. The intermixed region extends over substantially the entire bonded area of the layers.

In an exemplary embodiment, the resulting fusioned layers may have a structure as shown in FIG. 7E wherein the layers 101, 102 and 103 intermix over discrete areas 113 of the layers, thereby forming intermixed regions 113 and creating bonds at the interface 101-102 (between layers 102 and 101) and at the interface 102-103 (between layers 102 and 103). The fusion protrusions may act as separated bonding-regions between the layers. The fusion protrusions 113 may be arranged in two 2D arrays parallel with the layers.

In an exemplary embodiment, the materials 101, 102, and 103 can be made from the same chemical material/or substance but may have different structures (at micron level and/or millimeter level) and may be manufactured in different ways. For example, the outer layers 101 and 103 may be spun, woven, randomly deposited, or otherwise fibrous and porous in appearance, whereas the inner layer 102 may be continuous and impermeable. In this situation, applying heat and pressure specific to the same thermoplastic may cause the materials to melt together and bond via intermolecular forces.

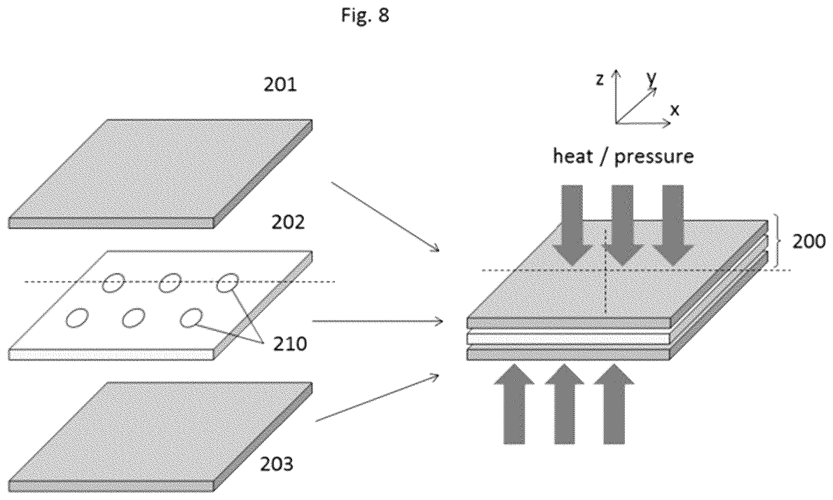

In an exemplary embodiment, a sleeve layer structure and a method for bonding together material layers into a sleeve layer material is described with respect to FIGS. 8 and 9A and 9B. This method can be used for material layers for which the heat and/or pressure based methods described with reference to FIGS. 6A and 6B and FIGS. 7A to 7E do not work for various reasons, such as because the porosity of the outer layers is not sufficient to support bonding by pressure and/or heat only. This method can be used for material layers which are incompatible with respect to bonding to each other. In this embodiment, the inner layer 202 includes a plurality of holes 210 which may be arranged in one line, several lines, or in a two dimensional matrix (as shown by FIG. 8).

The method of making a sleeve layer according to this embodiment may use three material layers 201, 202 and 203 as shown in FIG. 8. The layers 201 and 203 may be porous and may be made of the same material as the layers 101 and 103 described with reference to FIGS. 6-7. The layer 202 may be made of the same impervious material as the layer 102 in FIGS. 6-7. A plurality of holes 210 are formed in 202 (as shown in FIG. 8) by punching or other processes.

The method of making a sleeve layer according to this embodiment may include: a step of disposing the layer 202 in between the layers 201 and 203 (as shown in FIGS. 8 and 9A) followed by a fusion step. The fusion step may include applying pressure, heat, ultrasounds, light or a combination therein on the layers 201 and 203 such that part of layers 201 (shown by A in FIG. 9A) and part of layer 203 (shown by B in FIG. 9A above and under a hole 210 melt and fuse through the hole 210 such as to form a fusion region 220 as shown in FIG. 9B. The fusion may be performed such that the formed fusion region 220 covers and substantially seals the holes into the layer 202. While the fusion region 220 is formed by melting and fusion of materials 201 and 203 which may be porous, the fusion region 220 may not be porous and may form a substantially impermeable seal within the hole 210.

We note that without the fusion step the three layer structure (e.g. as shown in FIG. 9A) may not be impervious to gases and liquids since layer 202 has holes and layers 201 and 203 may be porous. The formed sleeve layer 200 in FIG. 9B is impervious because its inner layer includes a layer 202, made of impervious material, whose holes are covered and substantially sealed by the fusion region 220. The fusion regions 220 are continuous with the layers 201 and and may form an attachment between layers 201 and 203. The fusion region 220 may adhere to the layer 202 such as to form a seal and an attachment between layer 202 and layers 201 and 203.

In an exemplary embodiment, the melting of thermoplastic materials in region A of 201 and region B of 203 will fill in the holes/gaps 210, thereby creating an attachment between the layers 201, 202 and 203. In an exemplary embodiment the holes 210 in layer 202 used to make the sleeve material 200 may be disposed in a 2 dimensional matrix or array.

In another exemplary embodiment layers 201 and 203 may be made to maintain an impermeable barrier whereas layer 202 may be porous.

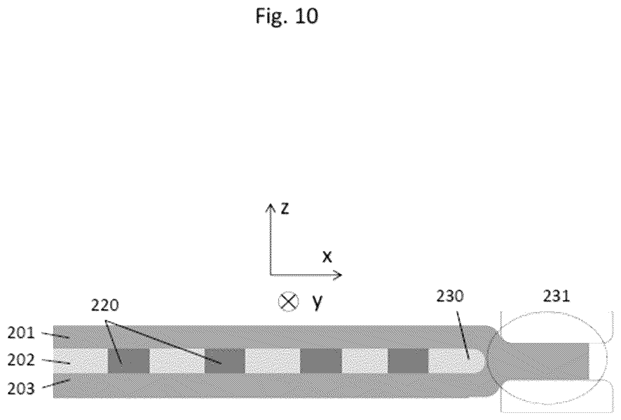

FIG. 10 shows an exemplary embodiment, in which the layer 201 and layer 203 may be fused together around layer 202 along an edge 230 of layer 202 thereby forming an end fusion region 231.

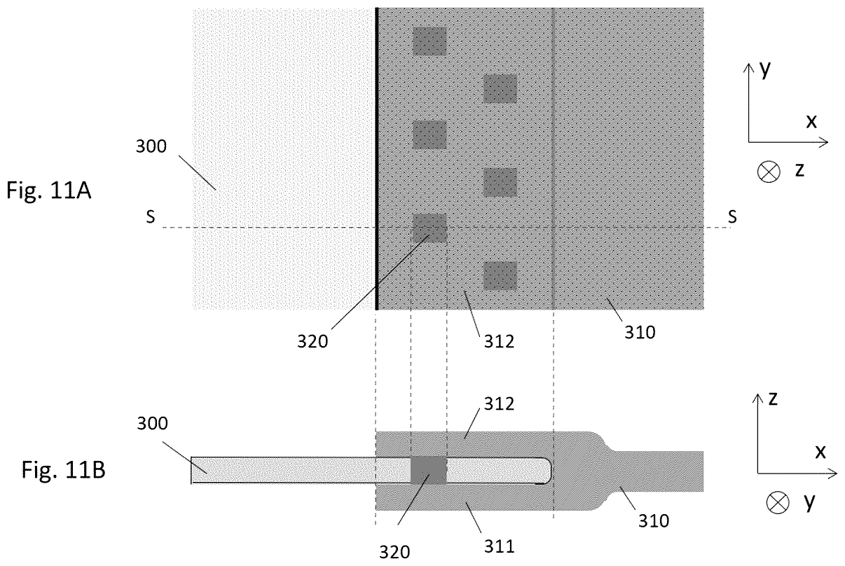

A method of attaching an edge of a layer 300 to an edge of a layer 310 is described with reference to FIGS. 11A and 11B. Layer 310 may include an upper branch layer 312 and a lower branch layer 311 (as seen in 11B). Layer 300 may include one or more rows of holes 320 similar to the holes 210 in FIGS. 8 and 9. FIG. 11A shows a view from above (along z) of the layers 300 and 310 attached to each other. FIG. 11B shows a cross section of the layers 300 and 310 in a plan “S” parallel with xz. The method may include on or more of the following steps: disposing layer 300 in between layers 311 and 312 as shown in FIG. 11B; attaching the layer 300 to layers and 312 by using the fusion methods described with reference to FIG. 9A and FIG. 9B.

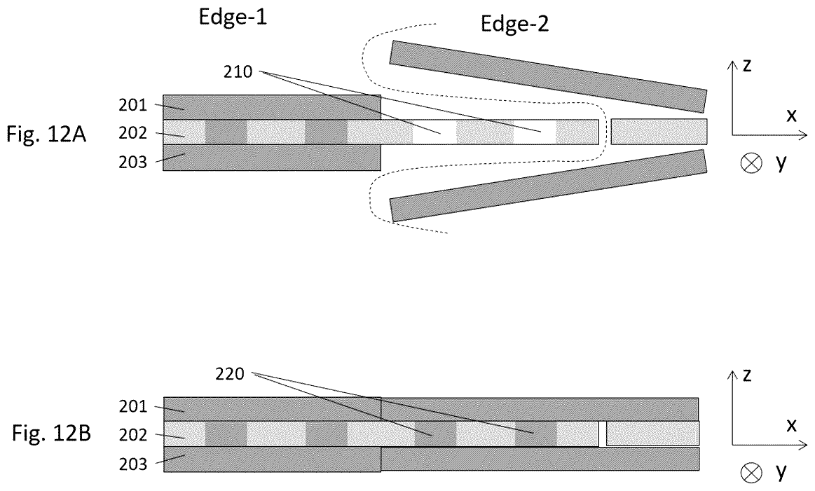

A method of attaching one edge of a sleeve to another edge of a sleeve is described with reference to FIGS. 12A and 12B. The method may include one or more of the following steps: preparing a first edge of a sleeve layer, such as the layers 200 or 100 described with reference to FIGS. 7-9, into the Edge-1 shown in the FIG. 12A; preparing a second edge of a sleeve layer, such as the layers 200 or 100 described with reference to FIGS. 7-9, into the Edge-2 shown in the FIG. 12A; disposing layer 202 of Edge-1 in between the layers 201 and 203 of Edge-2, wherein layer 202 includes one or more rows of holes such as the one described with reference to FIGS. 8-9; attaching the layer 202 of Edge-1 in between the layers 201 and 203 of Edge-2 by using the fusion method described with reference to FIG. 9.

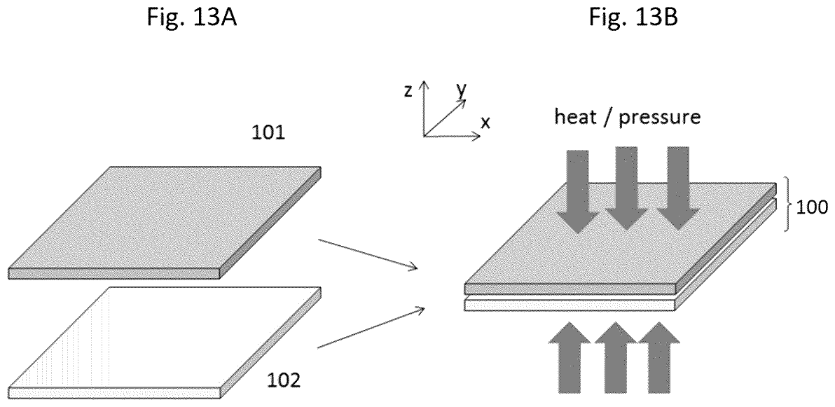

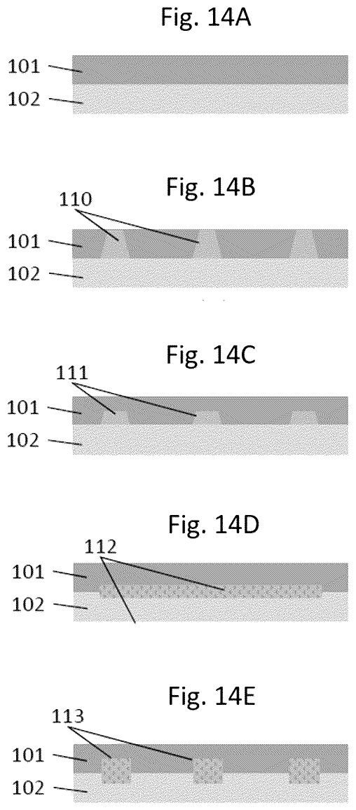

In an exemplary embodiment, the sleeve material layer 100 may include two distinct layers disposed over each other: layer 101 and layer and layer 102 (as shown in FIG. 13). Layer may be disposed to face the arms of the user, thereby touching the arms of the user during operation. Layer 102 may be disposed to face the inner of the flexible plastic enclosure, thereby being exposed to the environment inside the enclosure during operation. Layers 101 and 102 may be attached to each other as discussed hereinafter with reference to FIGS. 13 and 14.

The layers 101 and 102 may be adhered/bonded together by various means known by the skilled artisans, such as: applying heat, applying pressure, ultrasonic welding, laser welding, photochemical reactions (light induced), thermally induced reactions or any combination of them.

In an exemplary embodiment, a method of making a sleeve layer is described with reference to FIGS. 14A to 14E. In one embodiment, layer 102 may be an impervious thermoplastic whereas layer 101 may be made of a material including pores (which may not be impervious). Layers 101 and 102 may be adhered together by applying heat, specific to the melting temperature of the inner thermoplastic layer 102, and sufficient pressure, such as to allow for the thermoplastic to infuse (e.g. via melting) within the pores of the layer 101. In this way, the inner impervious thermoplastic will remain continuous with itself while also becoming mechanically adhered within the pores of the material 101, effectively creating a fitted bond.

FIG. 14A shows a cross section of the layers prior to fusion. In an exemplary embodiment, the resulting fusioned layers may have a structure as qualitatively shown in FIG. 14B wherein the layer 102 has formed fusion protrusions 110 inside the porous layers 101, thereby creating a bond between layers 101 and 102. The protrusions 110 completely penetrate through the layer 101. The fusion protrusions 110 may be arranged in a 2D array parallel with the layers and disposed essentially in the plane of layers. The fusion protrusions may act as separated bonding-regions between the layers.

In an exemplary embodiment, the resulting fusioned layers may have a structure as qualitatively shown in FIG. 14C wherein the layer 102 has formed fusion protrusions 111 inside layer 101, thereby creating a bond between layers 101 and 102. The protrusions 111 reach inside layers 101 without completely penetrating it. The fusion protrusions 111 may be arranged in a 2D array parallel with the layer and disposed essentially in the plane of layers. The fusion protrusions may act as separated bonding-regions between the layers.

In an exemplary embodiment, the resulting fusioned layers may have a structure as qualitatively shown in FIG. 14D wherein the layers 101 and 102 intermix between them at the interface 101-102 over substantially the entire area of the layers, thereby forming an intermixed region 112 and creating a bond between layers 102 and 101. The intermixed region extends over substantially the entire bonded area of the layers.

In an exemplary embodiment, the resulting fusioned layers may have a structure as shown in FIG. 14E wherein the layers 101 and 102 intermix over discrete areas 113 of the layers, thereby forming intermixed regions 113 and creating bonds at the interface between layers 102 and 101. The fusion protrusions 113 may be arranged in a 2D array parallel with the layers. The fusion protrusions may act as separated bonding-regions between the layers.

Layer 101 may be made of a material which feels comfortable when touching operator's hand and such as to absorb/wick up moisture. Layer 102 may be configured to maintain an impermeable barrier from the internal sterile environment and the external environment. Layer may include on or more of the following materials: impermeable thermoplastics such as thermoplastic polyurethane, polycarbonate, acetal copolymer polyoxymethlene, acetal homopolymer polyoxymethylene, acrylic, nylon, polypropylene, polystyrene, or other thermoplastics that are sufficiently non-brittle to act as a cloth-like material. The layers may be configured to focus on ergonomics, ease of use, or functional properties. These layer's functional properties could include: material's ability to reduce heat retention, increase heat retention, wick up moisture, decrease friction, increase friction, or other properties that would benefit the comfort or functionality of the sleeve. Layer 102 may be made of a material such as to create a comfortable feel when touching a patient and such as to absorb/wick up moisture from inside the enclosure.

Layers 101 and 102 may be made of the same materials, may have the same properties and composition, or may be made from the same type of material layer. At least one of the layers and 102 may be made of porous materials. At least one of the layers 101 and 102 may be made of impervious materials such as to form an impermeable barrier. Layers 101 and 102 may include on or more of the following materials: impermeable thermoplastics such as thermoplastic polyurethane, polycarbonate, acetal copolymer polyoxymethlene, acetal homopolymer polyoxymethylene, acrylic, nylon, polypropylene, polystyrene, or other thermoplastics that are sufficiently non-brittle to act as a cloth-like material.

In an exemplary embodiment, the materials 101 and 102 may be made from the same chemical material/or substance but may have different structures (at micron level and/or millimeter level) and may be manufactured in different ways. For example, the layer 101 may be spun, woven, randomly deposited, or otherwise fibrous and porous appearance, whereas layer may be continuous and impermeable. In this situation, applying heat and/or pressure specific to the same thermoplastic may cause the materials to melt together and bond.

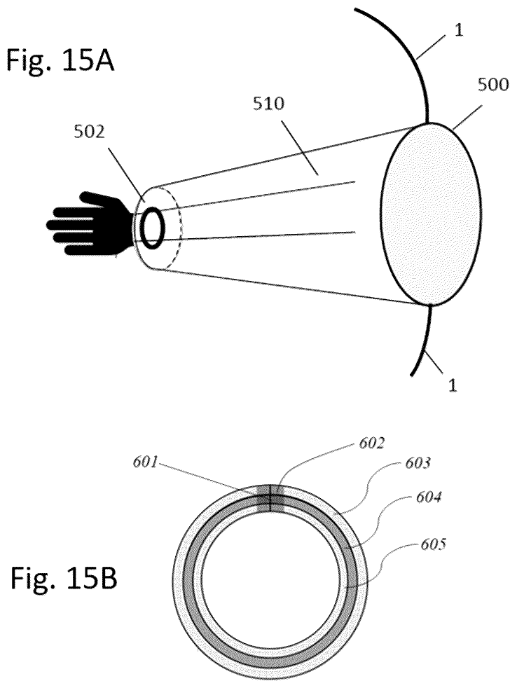

An exemplary embodiment is described hereinafter with reference to FIGS. 15A and 15B. The sterile enclosure 1 of the portable surgical system includes a port 500. The end of the sleeve 510 is attached to the port 500 such as to form a substantially impermeable seal as shown in FIG. 15A. The attaching of sleeve 510 to the port 500 is performed by sewing, thermal adhesion, or other processes known to the skilled artisan. The sterile sleeve 510 and the port 500 are configured to be used by an operator to access the sterile enclosure/environment.

The sleeve may further include a mechanism configured to secure the user's wrist or arm or hand in place with respect to the sleeve, such as: a strap, an elastic band, a string, or thread. The mechanism may further include a strap with an adhesive, velcro, or other friction based adhesion such as those found in athletic foam wraps. The strap may be tied off in a manner similar to that of sweatpants/scrubs, or wrapped then tied like a present.

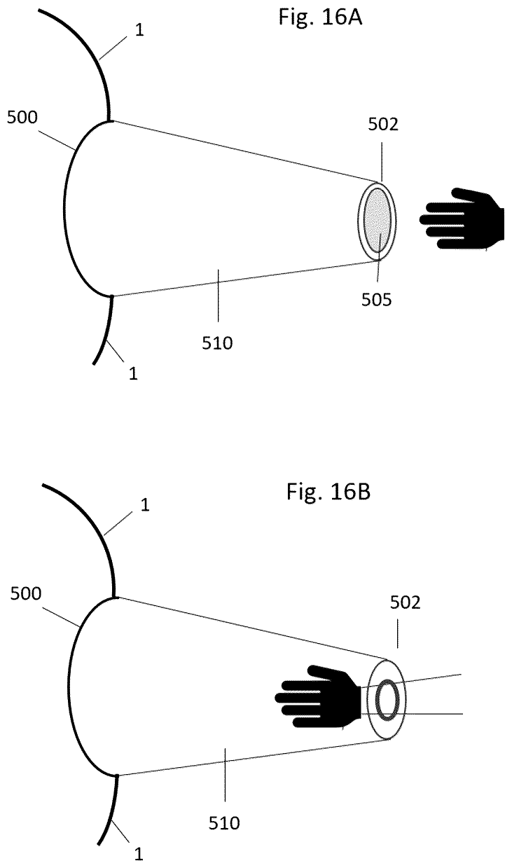

The sleeve may further include a removable sterile cover 505 (shown in FIG. 16A) configured to cover the opening 502 prior to use of the sleeve. The cover 505 is configured to be removed by operator prior to accessing the port and to cover or seal the port when the port is not used. The function of cover 505 is to retain the sterility of the internal portion of the sleeve. The cover 505 may be made as a disposable perforated element that can be torn off, a reusable cover that can be adhered to the end of the sleeve after proper sterilized preparation of the sleeve, or other adhesive or covering that can act as a barrier for port 500.

In one embodiment, the sterile sleeve may be constructed from one full layer sheet composite material such as, or similar to, the layers described with reference to FIG. 5-10. The edges of a full layer sheet are joined to form a conical shape, similar to the shape shown in FIG. 15A. FIG. 15B shows a cross-section of the sleeve in FIG. 15A at the level of the port 500. In an exemplary embodiment, the sleeve can be made by joining two edges of a sheet of material. The joining may be performed by thermally fusing the inner thermoplastic layer 604 (shown in FIG. 15B) and or by thermally fusing either or both layers 603 and 605 (shown in FIG. 15B), through methods as the ones described with reference to FIGS. 7-11, or through adhesive, or chemical bonding. Area 601 displays the terminal edges of the sterile sleeve material. Darkened area 602 displays where the aforementioned joining methods would be applied in this embodiment.

A method of using the portable surgical system is described hereinafter with reference to FIGS. 16A and 16B. Prior to using the sleeve, the cover 505 is covering the port 500 substantially sealing the inner of the enclosure from the outer environment as shown in FIG. 16A. The operator proceeds to remove the sterile cover 505. After the sterile cover 505 is removed, the operator inserts her hand in the sleeve through opening 502 as shown in FIG. 16B. The operator may be sterile to at least his or her wrist, prior to entering the sleeves. After the operator has placed his or her hand within the opening at 502, extra material can be tightened through an internally incorporated or external strap, elastic band, string, thread, or simply wrapped around the operator's wrist. Once secured, the operator will then invert the sleeve with their hand still secured, allowing the side of the sleeve to cover over their forelimb, thereby arriving at the configuration shown in FIG. 15A. In this embodiment, the sleeve side may be long enough to reach over the elbows, shoulders, or have additional slack beyond the length of the operator's forelimb. Once fully inverted, the operator may elect to place a fitted glove on and over their hand and wrist, effectively covering the point of entry 502 and further creating a better seal around the operator's wrist.

In an exemplary embodiment, the hand end of the sleeve may include a glove attached/continuous to the sleeve material and/or may not be open for hands to go through. In an exemplary embodiment the sleeves may be formed to have a shape which is different from a conical shape.

In exemplary embodiments, a sleeve layer structure and a method for bonding together material layers into a sleeve layer material is described with respect to FIGS. 8 and 9. These methods can be used for material layers for which the heat and/or pressure based methods described with reference to FIGS. 6 and 7 do not work for various reasons, such as because the porosity of the outer layers is not sufficient to support bonding by pressure and/or heat only. These methods can be used for material layers which are incompatible with respect to bonding to each other. These methods can also be used for material layers for which the heat and/or pressure based methods described with reference to FIGS. 6 and 7 are also used. In these embodiments, the inner layer 202 includes a plurality of holes 210 which may be arranged in one line, several lines, or in a two dimensional matrix (as shown by FIG. 8).

The methods of making a sleeve layer according to this embodiment may use three material layers 201, 202 and 203 as shown in FIG. 8. The layers 201 and 203 may be porous and may be made of the same material as the layers 101 and 103 described with reference to FIGS. 6-7. The layer 202 may be made of the same impervious material as the layer 102 in FIGS. 6-7. A plurality of holes 210 are formed in 202 (as shown in FIG. 8) by punching or other processes.

The methods of making a sleeve layer according to these embodiments may include: a step of disposing the layer 202 in between the layers 201 and 203 (as shown in FIGS. 8 and 9A) followed by a fusion step. The fusion step may include applying pressure, heat, ultrasounds, light or a combination therein on the layers 201 and 203 such that part of layers 201 (shown by A in FIG. 9A) and part of layer 203 (shown by B in FIG. 9A) above and under a hole 210 melt and fuse through the hole 210 such as to form a fusion region 220 as shown in FIG. 9B. The fusion may be performed such that the formed fusion region 220 covers and seals the holes 210 into the layer 202. While the fusion region 220 is formed by melting and fusion of materials 201 and 203 which may be porous, the fusion region 220 may not be porous and may form an impermeable seal within the hole 210.

We note that without the fusion step the three layer structure (e.g. as shown in FIG. 9A) may not be impervious to gases and liquids since layer 202 has holes and layers 201 and 203 may be porous. The formed sleeve layer 200 in FIG. 9B is impervious because its inner layer includes a layer 202, made of impervious material, whose holes are covered and sealed by the fusion region 220. The seal may be made such that liquids (e.g. blood, water, sweat) are prevented from passing through the holes. The fusion regions 220 are continuous with the layers and 203 and may form an attachment between layers 201 and 203. The fusion region 220 may adhere to the layer 202 such as to form a seal and an attachment between the layer 202 and the layers 201 and 203.

In an exemplary embodiment, the melting of thermoplastic materials in region A of 201 and region B of 203 will fill in the holes/gaps 210, thereby creating an attachment between the layers 201, 202 and 203. In an exemplary embodiment the holes 210 in layer 202 used to make the sleeve material 200 may be disposed in a 2 dimensional matrix or array.

In exemplary embodiments, the sleeve layer material may include both intermixed regions such as the ones described with reference to FIGS. 7 and (see e.g. intermixed regions 110, 111, 112 and 113 in FIGS. 7B to 7E and bonding/fusion regions such as described with respect to FIGS. 8-12 (see e.g. regions 220 in FIG. 9A). Such structure may have improved strength and/or sealing properties. Such structure may be easier to manufacture and/or may be less prone to defects.

In exemplary embodiments, the sleeve layer material may include intermixed regions such as the ones described with reference to FIG. 7E and bonding/fusion regions such as described with respect to FIGS. 8-12 (see e.g. regions 220 in FIG. 9A). In some embodiments, the intermixed regions may cover the entire surface area between the holes of regions 220. In some embodiments, the intermixed regions may cover an entire surface-area surrounding a certain number of holes 210 and regions 220 (e.g. 100 holes 210). Such structure may have improved strength and/or sealing properties. Such structure may be easier to manufacture and/or may be less prone to defects.

In this application the fusion regions (e.g. regions 220 in FIG. 9A) may also be referred as bonding regions. In this application the regions 110, 111, 112, and 113 in FIG. 7 may be referred as intermixed regions.

In addition to heat, pressure, ultrasound, and light, the layers may also be bonded by coextrusion, vacuum forming, and/or radio frequency welding.

The claimed inventions in this applications (e.g. materials, sleeves and methods of fabrication) are not limited by the specific embodiments and applications described herein. The sleeves, materials and methods described in this application can be used for applications different from the ones described in this application with respect to the portable surgical system. For example the sleeves and materials can be used for handling laboratory equipment, handling infants in hospital setting, handling chemicals and dangerous materials, electrical fabrication labs, sterile testing for immunocompromised subjects/specimens, cell/tissue culturing. The sleeves herein may be used for other applications, such as lens and optics testing and assembly, pharmaceutical prep, sterile environment for plant growth, etc.

The features of the invention disclosed herein, as specified by actual surgical end-users, distinguish it from prior art by enhancing usability, ergonomics, independence from external resources, and reliability under field conditions.

Although only a few embodiments have been described in detail above, those skilled in the art can recognize that many variations from the described embodiments are possible without departing from the spirit of the invention.

Embodiments of the invention are described herein with reference to figures and illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing.

The portable surgical systems disclosed herein may include alternate or additional sections which could be added based on procedural needs, such as to accommodate additional instrument trays or users. The above embodiments presented in this disclosure merely serve as exemplary embodiments and it will be apparent to those skilled in the art that various modifications and variations can be made in the present invention without departing from the spirit or scope of the invention.

It will be apparent to those skilled in the art that various modifications and variations can be made in the present invention without departing from the spirit or scope of the invention. Thus, it is intended that the present invention cover the modifications and variations of this invention provided they come within the scope of the appended claims and their equivalent.

The following publications are hereby incorporated by reference: [1] Teodorescu D L, Miller S A, Jonnalagedda S. SurgiBox: An ultraportable system to improve surgical safety for patients and providers in austere settings. IEEE Xplore GHTC 2017 (accepted, pending publication); [2] Teodorescu D L, Nagle D, Hickman M, King D R. An ultraportable device platform for aseptic surgery in field settings. ASME J Medical Devices. J. Med. Devices 10(2), (May 12, 2016); [3] Published international PCT application number PCT/US17/42266 filed on Jul. 14, 2017 and titled “Ultraportable System For Intraoperative Isolation and Regulation of Surgical Site Environment”.

1. A composite-material comprising:
two layers of a first material;
one layer of a second material;
wherein the layer of the second material comprises a plurality of holes; and
wherein the layer of the second material is disposed between the layers of the first material; and
a series of bonding regions extending, from one layer of the first material to the other layer of the first material, through the plurality of holes of the layer of the second material;
wherein the bonding regions are sealing the holes of the layer of the second material such as to form an impenetrable composite-material layer even when the layers of the first material are not impermeable;
wherein the bonding regions cause the layers to attach to each other.

2. The composite-material of claim 1, wherein the composite-material is manufactured by a process comprising:
applying heat, pressure, ultrasounds or light to the two layers of the first material; and
causing the two layers of the first material to fuse to each other through the holes.

3. The composite-material of claim 1, wherein the two layers of the first material are surrounding the layer of the second material at least over one edge of the layer of the second material and wherein the two layers of the first material are bonding with each other over the said edge.

4. The composite-material of claim 1, wherein at least one of the layers of the first material comprises pores or cavities and is not impermeable.

5. The composite-material of claim 1, wherein the composite-material is impermeable to a set of fluids, gasses, and substances comprising one or more of the following: blood, plasma, bacteria, viruses, dust particles, water, human and animal cells, and organic matter.

6. The composite-material of claim 1, wherein the layers of the first material have a set of ergonomical and functional properties comprising one or more of the following:
an ability to absorb blood and moisture,
an ability to absorb sweat off the arms of the operators,
an ability to create a comfortable feel on operator's arms, and
an ability to selectively allow the flow of a second set of gases and fluids; and
wherein the layers of the second material are lacking the said set of ergonomical and functional properties.

7. The composite-material of claim 1, wherein at least one of the first and the second materials is a thermoplastic.

8. The composite-material of claim 1, wherein the composite-material is shaped as a sterile sleeve comprising:
a substantially conically shaped layer made of the composite-material and configured to cover the length of an user's forelimb, the conically shaped layer comprising a first opening and a second opening;
wherein the first opening is configured to enable an user to insert a hand in the sleeve;
wherein the second opening is attached to a port of the enclosure such as to enable the hand of the user to access the inner of the enclosure via the port;
a mechanism configured to secure the user's wrist in place with respect to the sleeve such that a good seal and a personalized fit can be established.

9. The composite-material of claim 1, further comprising one or more intermixed regions between the layers of the first material and the layer of the second material;
wherein the intermixed regions act as a bond between the layers;
wherein the intermixed regions are formed by applying heat, pressure, ultrasound, or light or a combination of heat, pressure, ultrasound, light;
wherein the intermixed regions are different from the series of bonding regions extending, from one layer of the first material to the other layer of the first material, through the plurality of holes of the layer of the second material.

10. The composite-material of claim 9, wherein the composite-material is impermeable to a set of fluids, gasses, and substances comprising one or more of the following: blood, plasma, bacteria, viruses, dust particles, water, human and animal cells, and organic matter.

11. The composite-material of claim 9, wherein the layers of the first material have a set of ergonomical and functional properties comprising one or more of the following:
an ability to absorb blood and moisture,
an ability to absorb sweat off the arms of the operators,
an ability to create a comfortable feel on operator's arms, and
an ability to selectively allow the flow of a second set of gases and fluids; and
wherein the layers of the second material are lacking the said set of ergonomical and functional properties.

12. The composite-material of claim 9, wherein the one or more intermixed regions comprises a plurality of separated bonding-regions disposed in one or more 2 dimensional arrays,
wherein each of the 2 dimensional arrays is disposed at an interface between layers of the first material and layers of the second material.

13. The composite-material of claim 9, wherein the one or more intermixed regions combine into essentially one intermixed region extending over substantially the entire bonded area of the layers.

14. The composite-material of claim 9, wherein at least one layer of the first material comprises pores or cavities and is not impermeable.

15. The composite-material of claim 14, wherein the intermixed regions are the result of the penetration of material of one layer into the pores or cavities of another layer.

16. The composite-material of claim 9, wherein said composite-material has different functional or ergonomic properties on different sides.

17. A composite-material comprising:
one or more layers of a first material; and
one or more layers of a second material;
one or more intermixed regions between the layers of the first material and the layers of the second material;
wherein the intermixed regions act as a bond between the layers;
wherein the intermixed regions are formed by applying to the layers heat, pressure, ultrasound, light or a combination of them; and
wherein the intermixed regions are formed such as to maintain the continuity of the composite-layer and such that the composite-layer is impermeable.
